(19) United States
(12) Patent Application Publication
(10) Pub. No.: US 2010/0160882 A1
(43) Pub. Date: Jun. 24, 2010

(54) HYGIENE APPARATUS, PERSONAL MONITORING SYSTEM, AND METHOD OF USE THEREOF

(76) Inventor: William Lowe, Los Angeles, CA (US)

Correspondence Address: TROJAN LAW OFFICES, 9250 WILSHIRE BLVD, SUITE 325, BEVERLY HILLS, CA, 90212, US

(21) Appl. No.: 12/719,806

(22) Filed: Mar. 8, 2010

Related U.S. Application Data
(63) Continuation-in-part of application No. 12/121,980, filed on May 16, 2008.

(51) Int. Cl. A61F 13/42; A61F 13/45

(52) U.S. Cl. 604/361; 604/385.14

(57) ABSTRACT

The present invention includes a hygiene apparatus, more specifically, the hygiene apparatus includes a housing and replaceable absorbent portion. The hygiene apparatus can include an electronic monitoring system and sensory feedback system. The absorbent portion and electronic monitoring system are retained within the housing. The electronic monitoring system includes an electronic portion which detects fluids and activates a sensory feedback signal to the user when fluids contact the system and can thereby warn the user before leakage of fluids beyond the article occurs. The user can remove and replace the absorbent cartridge accordingly. The electronic monitoring system can also include a biosensor to analyze the fluids discharged, process the analyzed information, and provide the physiological information regarding the fluids to the user. The electronic monitoring system can also transfer the information to a remote device so that the user can view the results of system's analysis.

### CROSS-REFERENCE TO RELATED APPLICATIONS

This application is a continuation-in-part application of and claims priority to U.S. application Ser. No. 12/121,980, filed on May 16, 2008, which is hereby incorporated by reference in its entirety.

### FIELD OF THE INVENTION

The present invention relates to a hygiene apparatus. More specifically, the invention relates to a hygiene apparatus, including an electronic and personal monitoring system, and a method of use thereof.

### BACKGROUND OF THE INVENTION

Absorbent articles designed to absorb fluids discharged from the human body, such as sanitary napkins and panty liners, are well known. The main purpose of the absorbent articles in the prior art is to absorb menstrual discharge. Manufacturers of the sanitary napkins focus on developing thin and super absorbent materials to prevent overflow, leaks and resulting stains. Many different constructions of absorbent articles have been made to protect a user's clothing from being soiled by discharge of such fluids. The absorbent articles described in the prior art do not employ an electronic monitoring system to monitor absorbed fluids and provide feedback to users when the absorbent article has reached, or is approaching, capacity. Nor do absorbent articles in the prior art include an electronic monitoring system to analyze characteristics of the absorbed fluids and provide feedback to users concerning the users' health.

Absorbent articles in the prior art employ non-electronic methods to prevent leakage. Sanitary napkins with wings extending from the side edges of the pad are well known in the art. Another method to prevent side leakage is described in U.S. Pat. No. 5,817,079, in which discreet areas of fluid-repellent materials are placed in the sanitary napkin to provide barriers to bodily fluid leakage from the product. Similarly, Canadian Patent No. 884,608 describes treating the edges of a sanitary napkin with hydrophobic material to prevent side leakage. U.S. Pat. No. 5,624,423 describes a sanitary napkin that includes a barrier means encircling an absorbent protuberance to prevent leakage. These prior art references help to prevent leakage but do not teach sanitary napkins that warn users before leakage occurs.

Other prior art references describe absorbent articles that detect fluids. U.S. Pat. No. 5,647,863 describes a sanitary napkin that provides a signal by way of an indicator member that becomes noticeably stained when the storage capacity of the sanitary napkin is substantially exhausted. This type of fluid detection does not provide a specific and clear signal to the user that fluid has reached the edge of the sanitary napkin, warning the user that leakage may occur.

U.S. Pat. No. 5,728,125 describes a sanitary napkin having a temperature-sensitive reactive chemical product that responds by turning cold when it contacts and dissolves in menstrual flow. This sanitary napkin's fluid detection, however, is activated when any fluid reaches the pad, and only indicates to the user that her menstrual flow has begun. The fluid chemical product is not strategically placed on the perimeter of the pad and therefore cannot warn the user before fluids reach the edge of the pad to prevent leakage over the edge of the pad.

What is needed is a sanitary napkin having an electronic monitoring system to detect and measure fluids absorbed and provide feed back to the user regarding the amount of fluid absorbed to prevent leakage. Such an absorbent article will provide a greater peace of mind to women, as the electronic monitoring system will provide reliable feedback to women as to whether or not the absorbent articles require changing before embarrassing leaks occur.

What is also needed is an absorbent article with an electronic monitoring system that analyzes characteristics of the absorbed fluids to provide feedback to the user regarding the user's physiological conditions and/or health. The absorbent article with the electronic monitoring system employs an integrated biosensor array to measure, detect, and analyze characteristics of discharged fluids such as volume, oxygen, glucose, and other physiological indicators. The biosensor array can also detect nutrient deficiencies and cholesterol (HDL/LDL) levels, as well as provide for bacteria, virus, and/or cancer screening. The electronic monitoring system can store such information and transfer the stored data to a personal monitoring device, which has specialized software to review the data. The absorbent article will provide a means for a woman to monitor health and/or physiological conditions during menstruation that has not been provided by absorbent articles in the prior art. Such monitoring can reveal patterns in the woman's health history. Based on the history, the electronic monitoring system can suggest possible corrective health care measures or direct the user to share the information with a physician.

With these goals in mind, the inventor sought to create a hygiene apparatus that incorporates an electronic monitoring system to provide both a means to monitor and report a user's physiological characteristics, as well as a means to monitor and report the amount of fluids absorbed, and thereby prompt the user to take action (inspect, change the article, etc.) to prevent leakage.

### SUMMARY OF THE INVENTION

The present invention describes a hygiene apparatus. The hygiene apparatus can comprise a housing and a replaceable absorbent portion for absorbing bodily fluids, wherein the replaceable absorbent portion removably attaches with the housing. A user can replace the replaceable absorbent portion to prevent leakage of absorbed bodily fluids beyond the hygiene apparatus. The housing can comprise a retaining means for retaining said absorbent portion. The housing can also include a top side and a bottom side, wherein the top side and bottom side are removably attached with one another. The housing can also have integrated air vents.

The absorbent portion of the apparatus can comprise a retainer, wherein the retainer can removably attach with the housing. The absorbent portion can also include a raised protuberance, whereby a user can position the raised protuberance adjacent to a user's body to quickly absorb said bodily fluids into said absorbent portion.

The hygiene apparatus can be comprised of smart fabric technology to repel bacteria and virus formation. The smart technology can also be used for electronic applications in the hygiene apparatus. The apparatus can also include a thermal pack.

In another embodiment, the hygiene apparatus can include an interchangeable layer, wherein the interchangeable layer further comprises an endothermic or exothermic material, whereby the material is activated when bodily fluids reach the interchangeable layer and provides a temperature change signal to a user to warn the user when said bodily fluid reaches said interchangeable layer.

The different embodiments of the hygiene apparatus and personal monitoring systems that can include an electronic means and/or biosensor technology described below can include the aforementioned features of the hygiene apparatus set forth above, e.g., smart fabric technology, a thermal pack, an interchangeable layer, etc.

In yet another embodiment, the hygiene apparatus can include a housing, a replaceable absorbent portion, and an electronic monitoring system. The electronic monitoring system can comprise a fluid-activated sensor and an electronic means, wherein the fluid-activated sensor is electronically connected with the electronic means and transmits a signal to the electronic means when a bodily fluid contacts said fluid-activated sensor. The electronic means can provide a sensory feedback mechanism to a user of said hygiene apparatus upon receiving the signal from the fluid activated sensor. The sensory feedback mechanism prompts a user to replace the replaceable absorbent portion to prevent leakage of bodily fluids beyond the hygiene apparatus.

In another embodiment, a personal monitoring system is disclosed, comprising a hygiene apparatus having a housing, a replaceable absorbent portion, an electronic means, and at least one biosensor, wherein the biosensor is electronically connected with the electronic means and transmits information pertaining to a biological characteristic of a bodily fluid to the electronic means when a bodily fluid contacts a biosensor. The system includes a remote device having a display, wherein the remote device is electronically connected with the electronic means and the electronic means transmits the information received from the biosensor to the remote device. The remote device can display or store the information, and a user can thereby review the information.

In another embodiment, the personal monitoring system can include the hygiene apparatus providing a sensory feedback mechanism and at least one biosensor providing information pertaining to a biological characteristic of a bodily fluid.

The personal monitoring systems including at least one biosensor can include software to display, record, analyze, and/or organizing the information received from the biosensor. The system can also include a storage means to store the information.

The personal monitoring systems including an electronic means can also include at least one light-emitting diode, wherein each light-emitting diode is electronically connected with the electronic monitoring system to convey a status of the hygiene apparatus.

In another embodiment, the personal monitoring systems including an electronic means can include at least one radio-frequency identification tag and/or a power source to power the electronic monitoring system.

In yet another embodiment, the apparatus or systems including an electronic means can also include a vibration motor and/or vibration conduit to provide a vibrating sensory feedback signal to the user when fluids reach the fluid-activated sensors. In alternative embodiments, an air bladder that inflates when fluids reach the fluid-activated sensor can be included. In another embodiment, an electronic temperature change element can be included to provide a temperature change signal to the user when fluids reach the fluid-activated sensor.

In yet another embodiment, the hygiene apparatus and/or personal monitoring systems including an electronic means can include a remote device. When fluids reach the fluid activated sensors, the electronic means provides an auditory signal and a visual signal via the remote device.

Methods for using the hygiene apparatus and personal monitoring system are also described.

All embodiments of the hygiene apparatus and electronic transfers described herein can utilize electronics known in the art, including, but not limited to, electronic contact relays, signal processors and circuit boards or hardware known in the art, radio, universal serial bus (USB), Bluetooth™, Wi-Fi, or other wireless communication technology, and/or smart fabric technology.

### BRIEF DESCRIPTION OF THE DRAWINGS

FIG. 1A is a top perspective view of a hygiene apparatus according to the present invention;

FIG. 1B side orthogonal view of a hygiene apparatus according to the present invention;

FIG. 1C is a perspective view of an absorbent portion of a hygiene apparatus according to the present invention;

FIG. 2A is a perspective view of a biosensor array of a hygiene apparatus according to the present invention;

FIG. 2B is a side orthogonal view of a biosensor array of a hygiene apparatus according to the present invention;

FIG. 3 is a top orthogonal view of an electronic means and framing unit of a hygiene apparatus according to the present invention;

FIG. 4 is an exploded view of an embodiment of a hygiene apparatus according to the present invention;

FIG. 5A is a top orthogonal view of a hygiene apparatus according to the present invention, having the bottom section of the hygiene apparatus folded over;

FIG. 5B is an exploded view of an embodiment of a hygiene apparatus according to the present invention;

FIG. 6 is an exploded side view of an embodiment of a hygiene apparatus according to the present invention;

FIG. 7 is a top perspective view of an embodiment of a hygiene apparatus according to the present invention;

FIG. 8 is a top orthogonal view of an embodiment of a hygiene apparatus according to the present invention;

FIG. 9 is a side perspective view of an embodiment of a hygiene apparatus according to the present invention;

FIG. 10 is a top perspective view of an embodiment of a hygiene apparatus according to the present invention; and

FIG. 11 is an illustration of a method of using a hygiene apparatus and personal monitoring system according to the present invention.

### DETAILED DESCRIPTION OF THE INVENTION

The present invention includes a hygiene apparatus (1), more specifically, a feminine hygiene product, such as a sanitary napkin or panty liner, having an electronic monitoring system (12) that detects fluids absorbed by the apparatus (1) and provides a signal to the user when fluids have reached the system (12) and thereby warn the user before leakage beyond the hygiene apparatus (1) occurs. The system (12) can also analyze characteristics of fluids absorbed by the apparatus (1) and provide the results of the analysis to the user. The hygiene apparatus (1) can be in the form of other products, including incontinence pads. The apparatus (1) can be in the form of an attachment, insert, or can be integrated into undergarments. The apparatus (1) can have modular attachments and/or inserts which provide additional functionality, as discussed below.

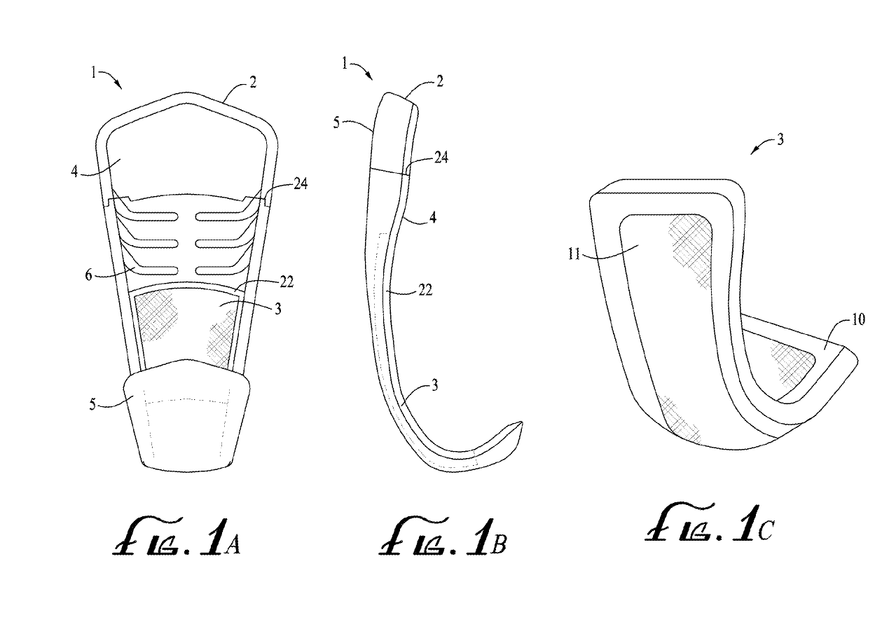

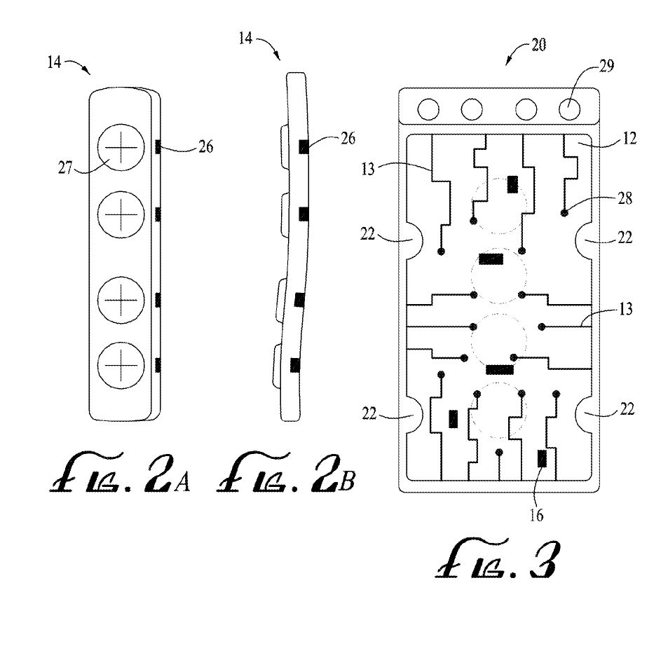

The apparatus (1) can have various shapes to contour the body and undergarments, for example, thong, extra-long, or styles with or without extra-protective side wings. The different shapes are provided to accommodate the individual user's needs. The hygiene apparatus (1) can also be in the form of a breast pad to absorb excess bodily fluids discharged during breastfeeding. Several embodiments of the hygiene apparatus (1) are illustrated in FIGS. 1A, 4, 5A-5B, and 6-10.

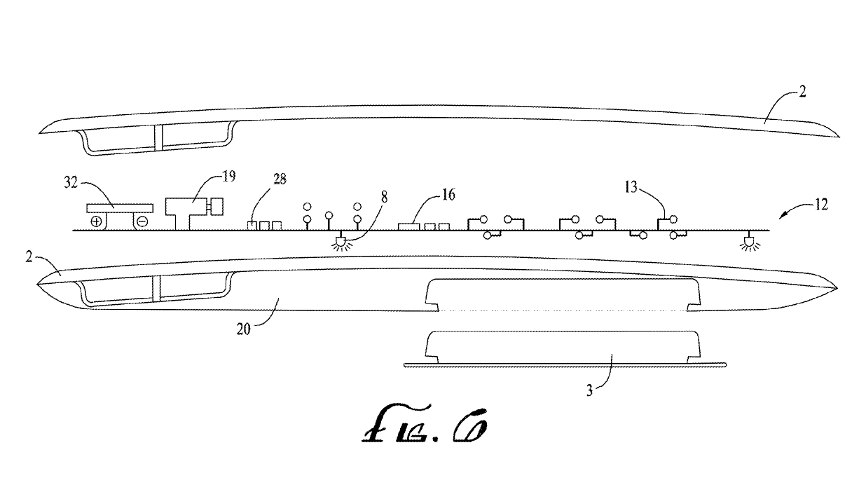

In a basic embodiment, the hygiene apparatus (1) comprises a reusable housing (2) and a replaceable absorbent portion (3). This embodiment is shown in FIGS. 1A-1C. The housing (2) of the hygiene apparatus (1) can retain the replaceable absorbent portion (3). To retain the absorbent portion (3) in the housing (2), the housing (2) can also include a retaining means (22), such as a lip, tabs, clips or other retaining means. Embodiments illustrating a retaining means are shown in FIGS. 1A-1B, 3 and 5B. The retaining means (22) can also comprise a sealed interaction with the housing (2), such as a seal through static cling, adhesive material, or a press/tolerance fit, where the absorbent portion (3) and housing (2) fasten together by friction when the two are pushed together, by relying on tensile and compressive strengths of the housing (2) and absorbent portion (3). In such embodiments, the absorbent portion (3) can include retainer (35) to create a reversible seal between the absorbent portion (3) within the hygiene apparatus (1). The retainer (35) can be in the form of a frame-like flexible cradle, as shown in FIG. 4.

As shown in FIGS. 1A-1B, the housing (2) has a top, body-facing side (4), and a bottom garment-facing side (5). The housing can be formed as a single unit, or can have the top body-facing side (4) removably attached with the bottom garment-facing side (5). The top, body-facing side (4) can further comprise a retaining means (22) or a framing unit (20) to retain the hygiene apparatus's components, such as the absorbent portion (3), and to provide additional perimeter leakage protection. The bottom side (5) can include a layer of an adhesive material or other suitable material to allow for removable attachment with an article of clothing. Alternatively, the hygiene apparatus (1) can be integrated into undergarments, in which the top side (4) and bottom side (5), or only the bottom side (5), of the hygiene apparatus (1) is incorporated into the material of the undergarment.

Also shown in FIG. 1A, the housing (2) can include air vents (6). The air venting channels (6) optimize the temperature regulation between the housing (2) of the hygiene apparatus (1) and the clothing to which it can be attached. The vents (6) ensure that a certain amount of space is maintained between the clothing and hygiene apparatus (1). The material can be structured to allow air to flow through the vents (6). Airflow can be directed up and around the bottom side (5) of the hygiene apparatus (1) to draw in cool air and direct warm moist air away from the body. The air vents can be integrated in the top side (4), bottom side (5), or both top and bottom sides of the housing (2).

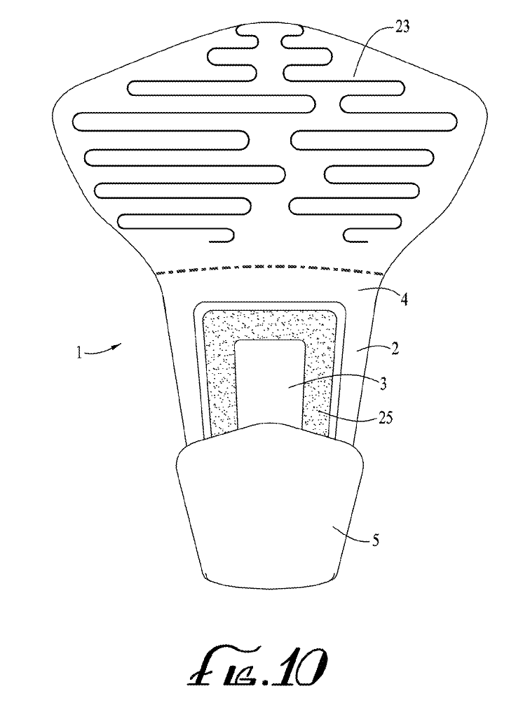

The reusable housing (2) can be anatomically contoured and made of a lightweight, washable molding. The housing (2) can be comprised of liquid silicone rubber, thermoplastic rubber, synthetic polymer, plastic polymer, suitable biogel or bioplastic materials, smart fabric, nonwoven elastic fibers or polymers, and/or other suitable materials known in the art. The non-woven material provides exceptional comfort, and can be formed to contour the female anatomy to ensure a better fit and comfort. The housing (2) can also be comprised of material known in the art that provide for active forming and molding based on the user's body temperature. As shown in FIG. 9, the housing (2) can be in a flexible shape that can be maintained in a folded or unfolded position. The housing (2) can also be comprised of smart fabric to repel bacteria, as discussed herein.

The absorbent portion (3) of the hygiene apparatus (1) can be disposable, replaceable and biodegradable. The absorbent portion (3) can be comprised of super absorbent polymers (SAP) to allow for maximum absorption of discharged fluids. The SAPs allow for use of a smaller absorbent pad while providing for greater absorption than that provided by larger pads known in the art. The absorbent portion (3) can utilize the construction methods and include features of sanitary napkins currently known in the art, such as a quick drying top layer, odor-absorbing properties, and can be made with different absorption properties depending on the level of absorption desired by the user. The absorbent portion (3) can include a scented or lotion infused element to reduce unpleasant odors and dryness. The absorbent portion (3) can also be comprised of water-soluble synthetic polymer such as polyvinyl alcohol that remains intact while in contact with discharged fluids but breaks down upon contact with water and continues to break apart when flushed, completely degrading in approximately a month. The absorbent portion (3) can include tab(s) (9) and/or a retainer (35) for easy-pull removal and replacement, as shown in FIG. 4.

As a non-limiting example shown in FIG. 1C, the absorbent portion (3) can include an outer core (10) and inner core (11). This construction creates a dual-channel fluid distribution to promote dryness by directing menstrual flow away from the body to the inner core. The outer core (10) can be comprised of a less permeable material than the inner core (11) to impede the flow of liquids from the core (11) to beyond the absorbent portion (3). The outer core (10) therefore provides an extra barrier to help prevent leakage.

The absorbent portion (3) can have through-holes, flow channels, or collection zones designed to direct bodily fluids toward the electronic monitoring system (12) and allow the fluids to come into contact with fluid activated sensors (28) and/or biosensors (14), for proper integration and functionality of the hygiene apparatus (1) that includes an electronic monitoring system (12). The electronic monitoring system (12) can also be integrated within the absorbent portion (3) to allow it to operate as designed and freely interact with the article's integrated components described herein.

Additionally, as shown in FIG. 4, the absorbent portion (3) can include tab(s)(9) and/or a retainer (35) for handling, easy-pull removal, replacement, ejecting, and/or positioning the absorbent portion (3) within the hygiene apparatus (1). The retainer (35) can form a frame-like flexible cradle to create reversible seal with the housing (2), as described above. The frame or retainer (35) can also have through-holes, flow channels, or collection zones to direct bodily fluids toward the electronic monitoring system (12) and allow the fluids to come into contact with fluid activated sensors (28) and/or biosensors (14), for proper integration and functionality of the hygiene apparatus (1) that includes an electronic monitoring system (12). The retainer (35) can also include an integrated electronic monitoring system (12).

Another embodiment of the absorbent portion (3) can include a raised protuberance (38), also shown in FIG. 4. The raised protuberance (38) can be located in the center of the absorbent portion (3) and is designed to fit into the vaginal cavity to allow the absorbent portion (3) to quickly absorb and wick the bodily fluids into the absorbent portion (3). The raised protuberance (38) operates as a tampon-like feature of the absorbent portion (3).

Embodiments of the apparatus (1) including an electronic monitoring system (12) are described in detail below. The embodiments of the apparatus (1) that include an electronic monitoring system can include the aforementioned features of the hygiene apparatus (1).

The electronic monitoring system (12) of the hygiene apparatus (1) can be integrated in the housing (2), the absorbent portion (3), comprise a separate component or layer of the hygiene apparatus (1), or can be integrated in more than one of the aforementioned components of the hygiene apparatus (1). Alternative embodiments which illustrate the electronic monitoring system (12) include FIGS. 4, 5A, 5B, 6, 7, and 8.

As shown in FIG. 4, the electronic monitoring system (12) can be placed in the housing (2). The absorbent portion (3) can overlay the electronic monitoring system (12). An interchangeable layer (18) can surround the absorbent portion (3), and the framing unit (20) can overlay the interchangeable layer (18). As assembled, the absorbent portion (3) is surrounded by the framing unit (20) and interchangeable layer (18) while overlaying the electronic monitoring system (12) and within the housing (2). The framing unit (20) can also include a lip, tabs, clips or other means (22) to retain the absorbent portion (3) in the housing (2).

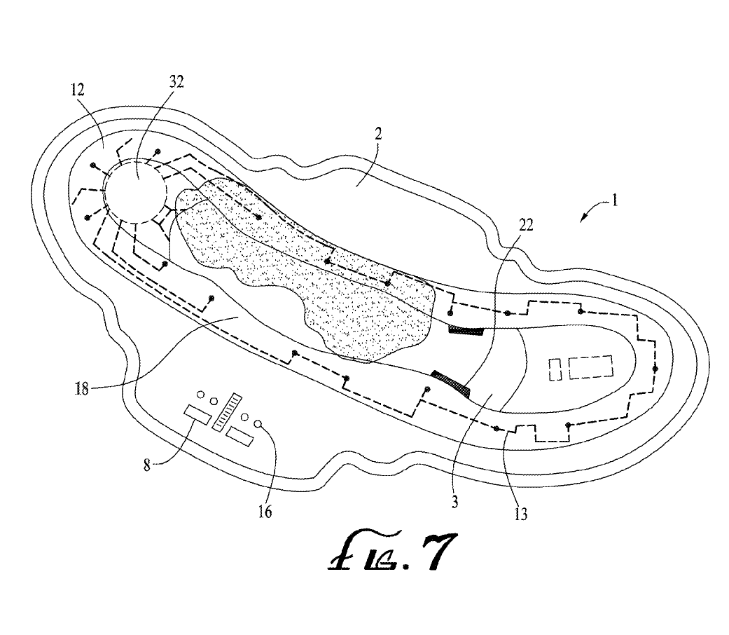

FIG. 5A shows the apparatus (1) assembled with the absorbent portion (3) placed within the housing (2), above the electronic monitoring system (12), and beneath the framing unit (20). As shown in FIG. 5B, the electronic monitoring system (12) can be held in place with a framing unit (20) and an extending lip, tabs, or clips (22), which can also retain the absorbent portion (3) with an optional retainer (35).

The electronic monitoring system (12) includes at least one fluid-activated sensor (28) and an electronic means (13), both of which can include associated electronics to process and/or transmit electronic signals to one another. All associated electronics, electronic connections, signals, and/or data transfers between the apparatus or system's electronic components and electronic monitoring system (12) described herein can utilize electronics known in the art, such as electronic contact relays, signal processors and circuit boards or hardware known in the art, radio, universal serial bus (USB), Bluetooth™, Wi-Fi, or other wireless communication technology, and/or smart fabric technology. Smart fabric technology utilizes metallic, conductive yarns woven into fabrics to create electronic circuits made out of textiles. Further, the system's electronic components can be soldered directly onto the conductive yarns of the smart fabric.

The smart fabric technology is useful when used in this hygiene apparatus (1) because the smart fabric is washable and flexible. Smart fabric technology can also be utilized for non-electronic components of the hygiene apparatus (1) because smart fabric has anti-bacterial properties. The electrically charged metal particles in smart fabric are known to repel bacteria and viruses. The smart fabric technology will allow for maintaining freshness of the apparatus (1) and preventing buildup of bacteria because it is integrated in the apparatus (1) and forms a reusable, washable unit. The smart fabric technology can be integrated into any component of the apparatus (1), including the housing (2), absorbent portion (3), retainer (35), and electronic components, including the electronic monitoring system (12) and components thereof.

The fluid-activated sensors (28) can be electronically connected with an electronic means (13) through the aforementioned electronics. A user can place the apparatus (1) adjacent to a body part discharging fluid. As the absorbent portion (3) becomes saturated with fluid, the fluid comes into contact with fluid-activated sensors (28) located within, underneath, or surrounding the absorbent portion (3). When a bodily fluid contacts the fluid-activated sensor (28), the fluid-activated sensor transmits a signal to the electronic means (13). The fluid-activated sensors (28) and electronic means (13) can each be integrated in the absorbent portion's retainer (35), in an interchangeable layer (18), in the housing (2), or a combination thereof.

Once it has received the signal from the fluid-activated sensor (28), the electronic means (13) can process the signal received by the fluid-activated sensor (28) and provide a sensory feedback mechanism to the user. The sensory feedback mechanism informs the user that the absorbent portion (3) should be inspected, removed or replaced. A user can replace the absorbent portion (3) before leakage occurs when the user detects the sensory feedback mechanism. The sensory feedback mechanism can be provided by the electronic means (13) using the aforementioned associated electronics, including but not limited to electronic contact relays (29), Bluetooth™ or wireless technology (33), and/or smart fabric technology.

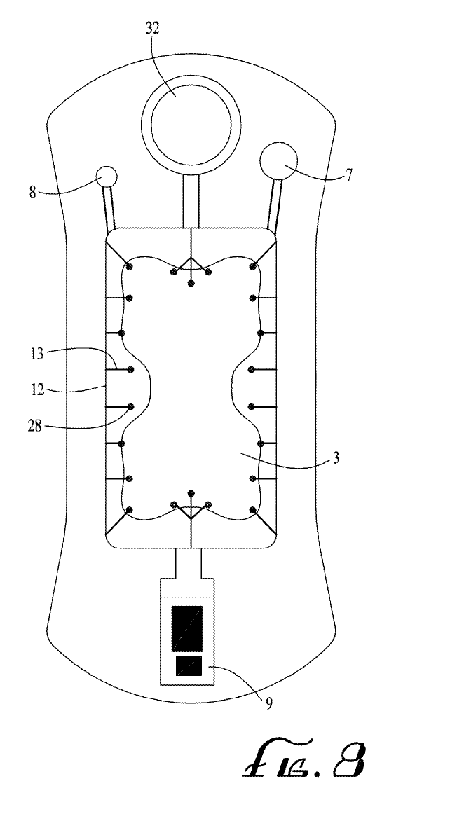

For example, in one embodiment, the electronic means (13) includes a micro-vibration motor (19). The micro-vibration motor (19) can be integrated in the interchangeable layer (18) of the hygiene apparatus (1), as shown in FIG. 4, and create vibrations throughout the apparatus (1) to discreetly notify the user inspect the apparatus (1) and prevent leakage. In this embodiment, discharged fluid contacts the fluid-activated sensors (28) of the electronic monitoring system (12) and the fluid activated sensors transmit a signal to the electronic means (13). The electronic means (13) receives and processes electronic signals from the fluid-activated sensors (28) to activate the vibration motor (19). Embodiments which use the vibration motor (19) can include a corresponding cavity (31) in the housing (2) to accommodate the motor (19). Alternatively, the vibration motor (19) can be integrated in the housing (2) of the hygiene apparatus (1), as shown in FIG. 8, or in the interchangeable layer (18), as shown in FIG. 6. The hygiene apparatus (1) can utilize micro or mini sized vibration motors known in the art.

The electronic means (13) can further comprise a vibration conduit or frame (36) electronically connected with the vibration motor. The vibration conduit (36) can be a separate unit or integrated with another component of the apparatus (1), such as the interchangeable layer (18) as shown in FIG. 5B. The vibration conduit (36) can transmit motor vibrations throughout the hygiene apparatus (1) when activated, and thereby alert the user of the article's status.

As shown in FIG. 4, the electronic means (13) of the hygiene apparatus (1) can include an inflatable/deflatable air bladder layer (21). A user can choose to use the interchangeable layer (18) with the vibration motor (19) or the interchangeable layer (18) with an air bladder (21) in the hygiene apparatus (1). Alternatively, the motor (19) and air bladder (21) can both be integrated into one interchangeable layer (18). In this embodiment, once fluid contacts the fluid-activated sensors (28), the fluid-activated sensors (28) send a signal to the electronic means (13). The electronic means (13) thereby activates the air bladder (21) in the interchangeable layer (18) to rapidly inflate or deflate as a warning signal of leakage that is about to occur. Alternatively, the interchangeable layer (18) can provide a means to manually inflate or deflate the air bladder layer for a more comfortable fit.

In another embodiment, the electronic means (13) can include an electronic temperature change element (30), such as an electric heating element. The fluid-activated sensors (28) will send a signal to the electronic means (13) when the qualified or specified amount of discharged fluids contacts the sensors (28). Once it receives the signal from the fluid-activated sensors (28), the electronic temperature change element (30) of the electronic means (13) provides a temperature change signal to the user. The temperature change element (30) can also comprise an electric cooling element that is activated by the fluid-activated sensors (28) as discussed above. An embodiment of the apparatus incorporating a temperature change element (30) is shown in FIG. 4.

The electronic means (13) can also process the signal received from the fluid-activated sensors (28) and transmit another signal to another component of the electronic means (13), apparatus (1), or remote device. For example, the electronic means (13) can transmit a signal to a remote device (15), such as a personal monitoring device or personal computer, which provides a sensory feedback mechanism when liquids reach the fluid-activated sensors (28). The apparatus (1) can use Bluetooth™ or wireless technology (33) to transmit the signal. The sensory feedback mechanism can be an audio or visual signal or message displayed on the remote device (15) to warn the user to change the absorbent cartridge before leakage occurs. The auditory or visual signals produced by the remote device (15) indicate to the user that the absorbent portion (3) should be inspected, removed or replaced. Once the absorbent portion (3) is replaced, the hygiene apparatus (1) system can be reset for its next use. The electronic means (13) can also transmit a signal to light-activated diodes (8) to display a visual sensory feedback mechanism that indicates the status of the apparatus (1).

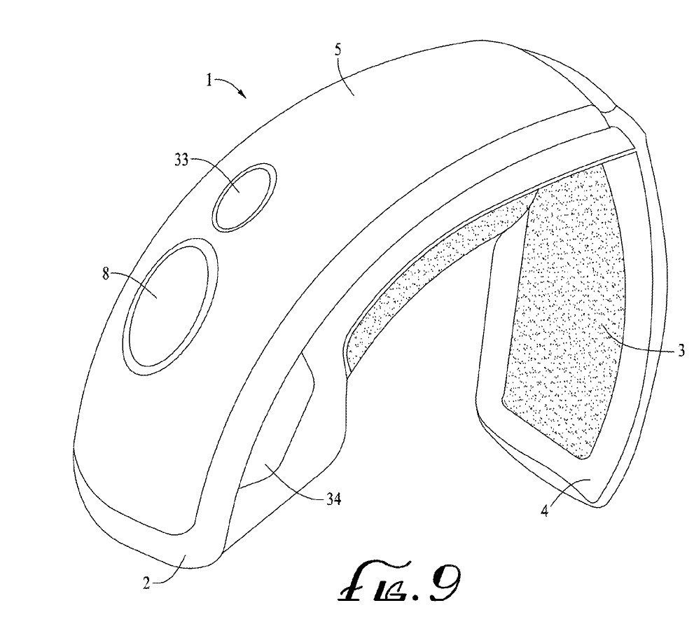

Alternatively, the hygiene apparatus (1) can include an interchangeable layer (18) comprising a material which provides feedback signals to the users without requiring activation by the fluid-activated sensors (28) and the electronic means (13). FIG. 7 shows the hygiene apparatus (1) according to this embodiment and including a housing (2), electronic monitoring system (12) layer, and interchangeable layer (18) without an electronic means (13).

When a bodily fluid, such as blood, reaches the interchangeable layer (18), the layer (18) is activated and provides a signal to the user. In one embodiment, the interchangeable layer (18) is comprised of an endothermic or exothermic material. In this embodiment, the interchangeable layer (18) reacts with the fluid that comes into contact with the material and provides a temperature change signal to the user. For example, an exothermic material, upon activation by bodily fluids, provides a warm sensation to the user, which is only perceived by the user. In the alternative, an endothermic material, upon activation by bodily fluids, provides a cold sensation to the user that is only perceived by the user. This specific location of the layer (18), which surrounds the absorbent portion (3), allows the hygiene apparatus (1) to provide a distinct signal to the user specifically when bodily fluids reach the periphery of the hygiene apparatus (1) and hence provides a distinct signal to the user before leakage can occur.

As known in the art, the endothermic or exothermic materials can be particles known in the art that absorb or release heat when dissolved in fluid. Also known in the art, the endothermic or exothermic materials can be substances that absorb or release heat upon reaction with an aqueous solution. The endothermic and exothermic materials can be bio-degradable, environmentally friendly and are non-hazardous materials known in the art. In another embodiment, the interchangeable layer (18) can include a band of exothermic material that is encapsulated by a dissolvable gel. When fluid reaches the gel casing, the gel casing dissolves and the band of exothermic material is exposed to the air and creates a thermal effect.

As a non-limiting example, the interchangeable layer (18) can also be comprised of a non-toxic material which provides a tingling sensory feedback mechanism to the user. The tingling sensation is activated when fluids reach the interchangeable layer (18) and thus warns the user to change the hygiene apparatus (1) before leakage occurs.

Overall, one can use any of the aforementioned embodiments of the hygiene apparatus (1) and personal monitoring system by positioning the hygiene apparatus adjacent to a user's body to absorb bodily fluids discharged from the body, detecting a sensory feedback mechanism when the discharged fluids contact the fluid activated sensor (28) of the hygiene apparatus (1), and replacing the absorbent portion (3) when the sensory feedback mechanism is detected. The user thereby avoids leakage of discharged fluids from the hygiene apparatus (1). Alternatively, the user can replace the absorbent portion (3) upon detecting a non-electronically fluid activated sensory feedback mechanism as discussed above, or can replace the absorbent portion at any time.

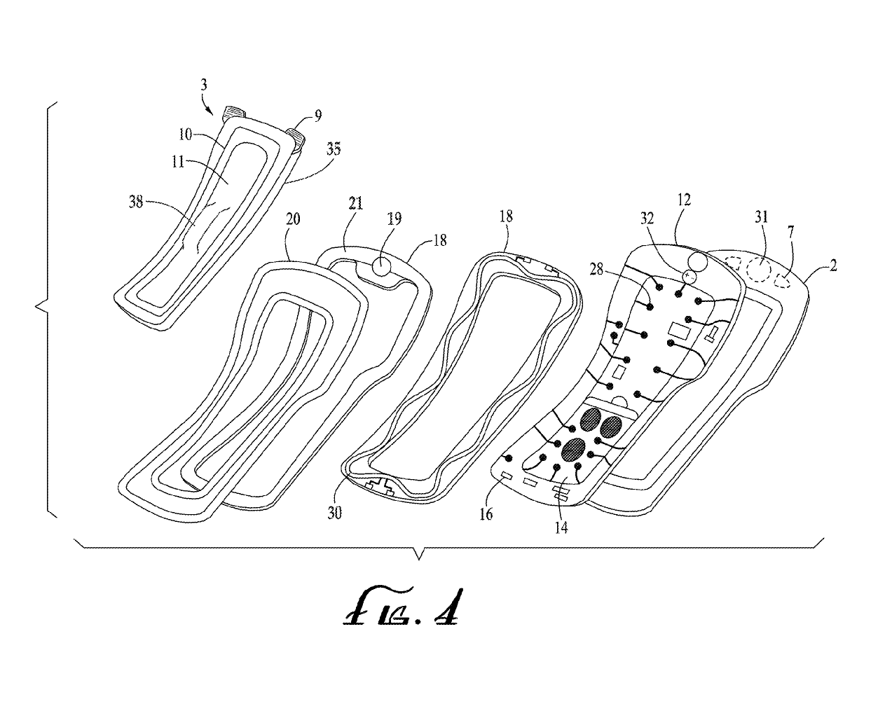

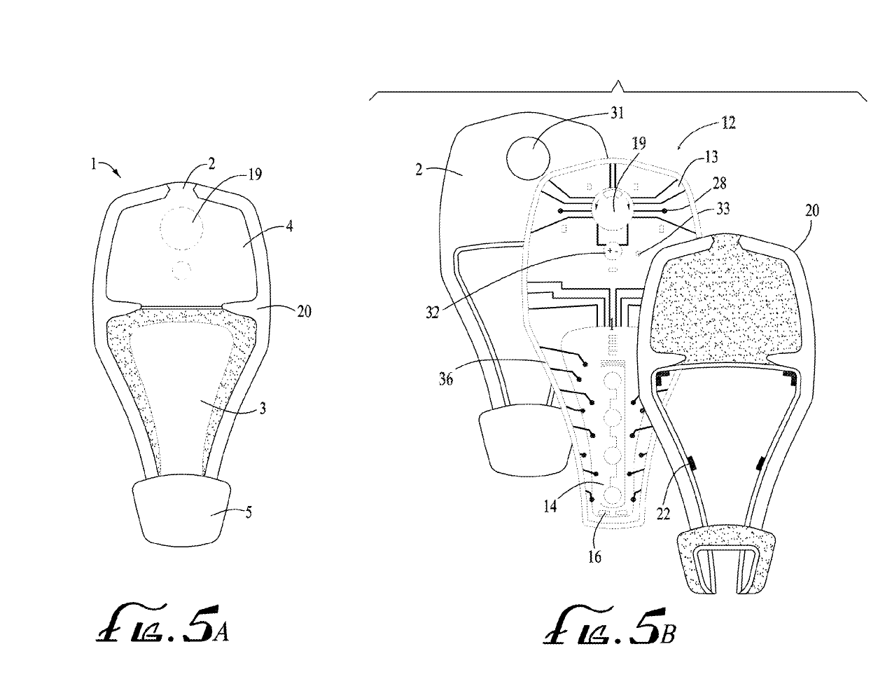

In another embodiment, the fluids that reach the electronic monitoring system (12) can be analyzed by using the biosensor array (14) as set forth below. The hygiene apparatus (1) can also include biosensor technology known in the art for analysis of discharged fluids, such as blood, sweat or urine. The biosensor technology can be comprised of an interchangeable biosensor array (14) comprised of one or more biosensors (27), illustrated in FIGS. 2A-2B. The biosensor (27) is an integrated device that includes a biological recognition element(s) (e.g., biological material such as tissue, microorganisms, organelles, cell receptors, enzymes, antibodies, nucleic acids, etc, or a biologically derived material, bioengineered material, or biomimic). As a non-limiting example, glucose oxidase enzyme can be used in the biosensor (27) to detect levels of glucose.

The biosensor (27) also includes a transducer or detector element. The transducer or detector element is capable of detecting the biological reaction(s) between the discharged fluid and the biological element(s), and converting the signal resulting from the reaction into another signal which can be processed, measured, or quantified. The transducer or detector element can use a physicochemical process, such as an optical, piezoelectric, or electrochemical method. To display the signal produced by the transducer or detector element of the biosensor (27) in a user-friendly way, the biosensor array (14) includes associated electronics or signal processors (26). The biosensor (27) can utilize the aforementioned associated electronics used by the electronic monitoring system (12), including, but not limited to, smart fabric technology. As shown in FIGS. 2A and 2B, multiple biosensors (27) comprise the biosensor array (14), which also includes the associated electronics or signal processors (26) to transfer the transducer signal to the electronic means (13) of the hygiene apparatus (1).

Using the biosensor array (14), the hygiene apparatus (1) can measure, detect and analyze characteristics of the discharged fluids such as the volume of fluids discharged, oxygen levels, glucose levels, triglyceride levels, hormone levels, and other physiological indicators of the fluids discharged. When fluids absorbed by the absorbent portion (3) come into contact with the biosensor array (14), analysis of the fluids is enabled because the fluids react with the biological element of the biosensor (27).

The fluids can come into contact with the biosensor array (14) when either the biosensor array (14) is integrated in the electronic monitoring system (12), within the absorbent portion (3), or exists as a separate component of the apparatus (1), underlying the absorbent portion (3). As known to one of ordinary skill in the art, a biosensor array (14) can be designed and created to detect nutrient and/or vitamin deficiencies, muscle contraction, cholesterol (high-density lipoprotein and low-density lipoprotein) levels, and/or hormone levels. The biosensor array (14) can also be designed and created to detect or screen for bacteria, viruses, and cancer. The user can use the biosensor array (14) to perform different analyses as described above based on the biosensor(s) (27) used, and the biosensor array (14) is interchangeable. The biosensor array (14) can be comprised of multiple biosensors (27) to perform different analyses at the same time.

The biosensor (27) can then transmit the information of the detected biological characteristic of the user to the electronic means (13) of the hygiene apparatus (1). The electronic means (13) of the electronic monitoring system (12) can temporarily store information derived from the biosensor array (14) and/or transmit the information to a remote device (15), such as a personal monitoring device that acts as a portable receiver. The electronic monitoring system (12) and electronic means (13) can include built-in Bluetooth™, Wi-Fi, Radio, universal serial bus (USB) (34), and/or wireless technology (33) known in the art to transfer or transmit data to a mobile remote device (15), such as a personal monitoring device, for review and analysis of the data and/or display the status of the electronic monitoring system (12). The system (12) can also use a storage means, such as a flash or microSD memory card or a similar storage medium, and corresponding reader, to store the data.

The remote device (15) can be carried or worn by the user and can be used to store and review the information received from the electronic monitoring system (12). The remote device (15), can be a portable or mobile device, such as a personal digital assistant (PDA), cell phone, pager, watch, or another wearable or clip-on device that displays the information received from the electronic monitoring system (12). The remote device (15) can also be less mobile or not worn, e.g., a desktop or laptop computer. As shown in FIG. 11, the remote device (15) is a mobile personal monitoring device that is worn on the wrist.

The remote device (15) can also be used to send the information received from the electronic monitoring system (12) of the hygiene apparatus (1) to another remote device (17) for review. For example, the analysis and measurement data obtained can be transferred from the remote device (15) or from directly from the electronic monitoring system (12) to another remote device (17), such as a computer, to be reviewed by the user and/or shared with a physician. The remote device (15) can include built-in Bluetooth™, Wi-Fi, Radio, and/or universal serial bus (USB) technology known in the art to transfer, transmit, or receive data with another device (17). The remote device (15) can be used to program and enter settings of the device, such as timer intervals, warning modes, etc and can include software to program the apparatus (1) or display information received by the electronic monitoring system (12).

Use of the hygiene apparatus (1) with an electronic monitoring system (12) in this manner is shown in FIG. 11. The user positions the hygiene apparatus (1) in proximity to the body part discharging fluid. For example, the hygiene apparatus (1) is attached to the user's undergarment such as underwear to collect vaginal discharge. The hygiene apparatus's electronic monitoring system (12) including an electronic means (13) with wireless technology (33) transmits the information stored in the electronic monitoring system (12) to the remote device (15). The user can view and review the information on a remote device (15), such as a mobile personal monitoring device, or can further transfer the information to another remote device (17), such as a computer, to view and review the information derived from the article's electronic monitoring system (12).

The personal monitoring system can utilize software designed for analysis, display, organization, and/or recordation of the information obtained from the system. The software application can display relevant physiological data and electronically send the obtained information with others, such as a physician. The system (12) can be used in conjunction with other health monitoring and training systems known in the art interpret the history of the data and possibly implement corrective action. The remote device (15) can also include a control interface to control functions of electronic monitoring system (12) of the hygiene apparatus (1) described herein. Like the electronic monitoring system (12), the remote device (15) can include a storage means, such as a flash memory card or similar storage medium, and corresponding reader to store the data collected from the electronic monitoring system (12). Overall, the hygiene apparatus (1) embodiment that includes a biosensor array (14) is capable of detecting, recording, and transmitting information regarding a physiological change or process.

A push button or touchpad feature (7) can be incorporated into the housing (2) or the electronic monitoring system (12) to control the electronic means (13) of the hygiene apparatus (1) via an electronic or wireless connection. The housing (2) can also include light-emitting diodes (LED) (8) to display the status of the electronic monitoring system (12). For example, the electronic means (13) can send a signal to the LED to display a visual signal that indicates that bodily fluids have reached the fluid-activated sensors (28). The LEDs (8) can also individually be programmed to light up in response to the electronic monitoring system's biosensor analysis to indicate results of the electronic monitoring system's biosensor analysis of the discharged fluids. For example, an LED (8) light can light up to indicate whether a user is hormonally imbalanced after the biosensor (27) analyzes hormone levels of a user's discharged fluids. Different LEDs (8) may be arranged on the housing (2) to indicate different analysis results. Embodiments of the hygiene apparatus (1) that include a housing (2) having the push button (7) functions and LED (8) lights are shown in FIGS. 7 and 8.

Radio-frequency identification (RFID) tag(s) (16) may be incorporated into the hygiene apparatus (1) or any of its components to identify the hygiene apparatus (1) or its components and process or transmit information obtained by the electronic monitoring system (12). For example, the RFID can be integrated in the housing (2), the absorbent portion (3) to indicate level of absorption (heavy, medium, medium or light), the biosensor (27) to indicate the type of fluid analysis (LDL, hormone, feedback sensor etc.), and/or the electronic monitoring system (12) to indicate which sensory feedback mechanisms are in place. The RFID information can be included as part of the article's review, feedback and reporting of processed information. For example, the RFID can be used to transmit the information received from the electronic monitoring system (12) to a remote device (15). The RFID can also provide interactive product instructions. Furthermore, the integrated RFIDs activate in-store product information or can be used with a retail store's automated inventory control management systems to indicate, for example, exactly which, how many and/or the location of each hygiene apparatus component, such as the housing, biosensor, absorbent cartridge and feedback sensory mechanisms etc., in stock at any given time. The RFID tags (16) can be micro-sized and/or flexible. Embodiments incorporating an RFID tag are shown in FIGS. 3-4, 5B, and 6-7.

The hygiene apparatus (1) with an electronic monitoring system (12) can include a framing unit (20). The framing unit (20) can be anatomically contoured and made of a lightweight, washable molding. The framing unit (20) can be comprised of liquid silicone rubber, thermoplastic rubber, synthetic polymer, plastic polymer, suitable biogel or bioplastic materials, and/or nonwoven elastic fibers or polymers known in the art. The non-woven material provides exceptional comfort, and can be formed to contour the female anatomy to ensure a better fit and comfort. The framing unit (20) can also be comprised of material known in the art that provide for active forming and molding based on the user's body temperature.

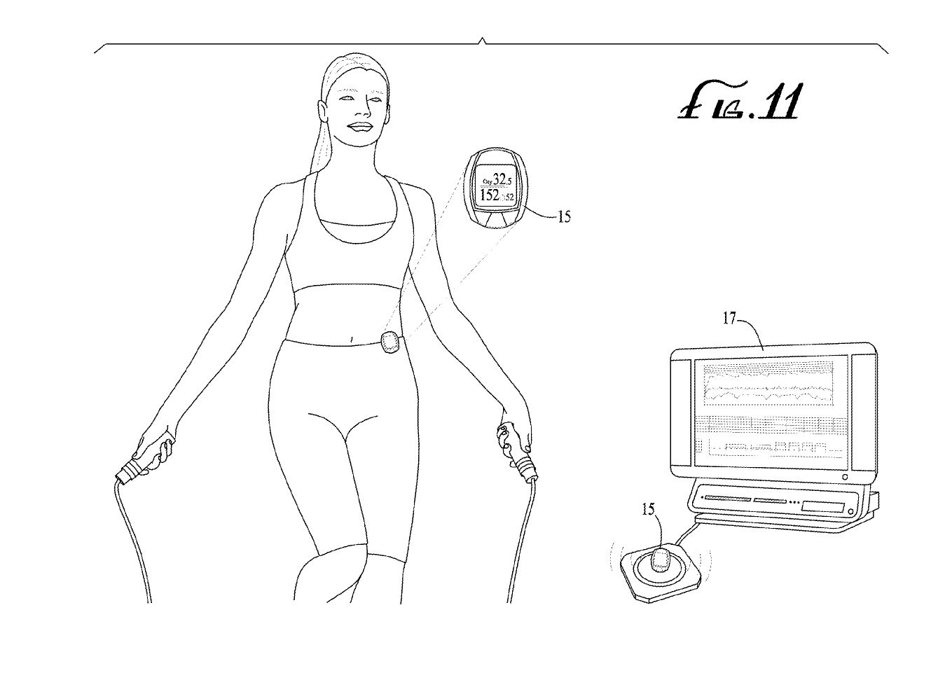

In another embodiment, the hygiene apparatus (1) can include an integrated thermal pack (23), as shown in FIG. 10. Alternatively, the thermal pack (23) can be a separate component and attach with the hygiene apparatus (1) via an attachment means (24) known in the art, such as a hook and loop attachment means. FIG. 1A illustrates a non-limiting example of a hygiene apparatus (1) having an attachment means (24) to attach a thermal pack (23) with the hygiene apparatus (1). The thermal pack (23) can be powered by battery, solar power, a universal serial bus (USB) (34) connection with the hygiene apparatus's electronic monitoring system (12), or use the technology of thermal packs known in the art, such as self-contained heating pads comprised of ingredients that are activated to provide hours of warm heat once exposed to air. Such self-heating thermal packs known in the prior art are comprised of ingredients such as iron, salt, charcoal and water, or other chemical compounds known to induce heating. The embodiment of the hygiene apparatus (1) with a thermal pack (23) may be used to help relieve menstrual cramps.

The hygiene apparatus (1) can have any of the aforementioned layers or components already integrated into the hygiene apparatus (1). Further, all embodiments described herein can include a biosensor (27) as part of the electronic monitoring system to analyze the fluids as set forth above. The hygiene apparatus (1) can therefore include either the sensory feedback mechanism or the biosensor technology, both the sensory feedback mechanism and the biosensor technology, or neither the sensory feedback mechanism nor the biosensor technology.

Various power sources (32) may be used to power the components of the hygiene apparatus (1) with an electronic monitoring system (12). The power source (32) of the hygiene apparatus (1) can include a battery, such as a rechargeable micro battery, a micro solar panel, or it can be powered by induction.

While the foregoing describes the present invention in relation to illustrations and examples, it is understood that it is not intended to limit the scope of the invention to the illustrations and examples described herein. On the contrary, it is intended to cover all alternative modifications and equivalents that may be included in the spirit and the scope of the invention as defined by the appended claims.

What is claimed is:

1. A hygiene apparatus, comprising:
a housing;
a replaceable absorbent portion;
an electronic monitoring system, wherein said system comprises a fluid-activated sensor and an electronic means, wherein said fluid-activated sensor is electronically connected with said electronic means and transmits a signal to said electronic means when a bodily fluid contacts said fluid-activated sensor, and wherein said electronic means provides a sensory feedback mechanism to a user of said hygiene apparatus upon receiving said signal,
whereby said sensory feedback mechanism prompts a user to replace said replaceable absorbent portion to prevent leakage of said bodily fluid beyond said hygiene apparatus.

2. A personal monitoring system, comprising:
a hygiene apparatus having a housing, a replaceable absorbent portion, an electronic monitoring system comprising an electronic means, and at least one biosensor, wherein said biosensor is electronically connected with said electronic means and wherein said at least one biosensor transmits information pertaining to a biological characteristic of a bodily fluid to said electronic means when a bodily fluid contacts said at least one biosensor; and
a remote device having a display, wherein said remote device is electronically connected with said electronic means and said electronic means transmits said information to said remote device, and wherein said remote device displays said information upon receiving said analysis from said electronic means,
whereby a user can review said information.

3. A personal monitoring system, comprising:
the hygiene apparatus of claim 1;
wherein said hygiene apparatus further comprises at least one biosensor, wherein said biosensor is electronically connected with said electronic means and wherein said at least one biosensor transmits information pertaining to a biological characteristic of a bodily fluid to said first electronic means when a bodily fluid contacts said at least one biosensor; and
a remote device having a display, wherein said remote device is electronically connected with said electronic means and said first electronic means transmits said information to said remote device, and wherein said remote device displays said information upon receiving said information from said electronic means,
whereby a user can review said information.

4. The personal monitoring system of claim 2, further comprising software to perform one or more functions selected from a group consisting of displaying said information, recording said information, analyzing said information, and organizing said information.

5. The personal monitoring system of claim 2, further comprising a storage means to store said information.

6. The hygiene apparatus of claim 1, further comprising at least one light-emitting diode, wherein each said light-emitting diode is electronically connected with said electronic monitoring system to convey a status of said hygiene apparatus.

7. The hygiene apparatus of claim 1, further comprising at least one radio-frequency identification tag.

8. The hygiene apparatus of claim 1, further comprising a power source to power said electronic monitoring system.

9. The hygiene apparatus of claim 1, wherein said electronic means further comprises a vibration motor, and wherein said sensory feedback mechanism comprises a vibrating signal,
whereby said vibrating signal prompts said user to replace said replaceable absorbent portion to prevent leakage of said bodily fluid beyond said hygiene apparatus.

10. The hygiene apparatus of claim 1, wherein said electronic means further comprises a vibration conduit, and wherein said sensory feedback mechanism comprises a vibrating signal,
whereby said vibrating signal prompts said user to replace said replaceable absorbent portion to prevent leakage of said bodily fluid beyond said hygiene apparatus.

11. The hygiene apparatus of claim 1, wherein said electronic means further comprises an air bladder, and wherein said sensory feedback mechanism comprises inflation of said air bladder,
whereby said inflation prompts said user to replace said replaceable absorbent portion to prevent leakage of said bodily fluid beyond said hygiene apparatus.

12. The hygiene apparatus of claim 1, wherein said electronic means further comprises an electronic temperature change element, and wherein said sensory feedback mechanism comprises a temperature change signal,
whereby said temperature change signal prompts said user to replace said replaceable absorbent portion to prevent leakage of said bodily fluid beyond said hygiene apparatus.

13. The hygiene apparatus of claim 1, wherein said electronic means further comprises a remote device, and wherein said sensory feedback mechanism is selected from a group consisting of an auditory signal and a visual signal,
whereby said sensory feedback mechanism prompts said user to replace said replaceable absorbent portion to prevent leakage of said bodily fluid beyond said hygiene apparatus.

14. A method for using the hygiene apparatus of claim 1, comprising the acts of:
positioning said hygiene apparatus adjacent to a user's body to absorb bodily fluids discharged from said user's body;
detecting said sensory feedback mechanism when said discharged fluids contact said fluid activated sensor of said hygiene apparatus; and
replacing said absorbent portion of said hygiene apparatus when said sensory feedback mechanism is detected, whereby a user avoids leakage of said discharged fluids from said hygiene apparatus.

15. A method for using the personal monitoring system of claim 2, comprising the acts of:
positioning said hygiene apparatus adjacent to a user's body to absorb bodily fluids discharged from said user's body; and
reviewing said information of said biosensor via said remote device.

16. A hygiene apparatus, comprising:
a housing;
a replaceable absorbent portion for absorbing bodily fluids, wherein said replaceable absorbent portion removably attaches with said housing,
whereby a user can replace said replaceable absorbent portion to prevent leakage of said bodily fluids beyond said hygiene apparatus.

17. The hygiene apparatus of claim 16, wherein said housing comprises a retaining means for retaining said absorbent portion.

18. The hygiene apparatus of claim 16, wherein said housing comprises a top side and a bottom side, wherein said top side and said bottom side are removably attached with one another.

19. The hygiene apparatus of claim 16, wherein said housing has air vents.

20. The hygiene apparatus of claim 16, wherein said absorbent portion comprises a retainer, wherein said retainer removably attaches with said housing.

21. The hygiene apparatus of claim 16, wherein said absorbent portion further comprises a raised protuberance, whereby a user can position said raised protuberance adjacent to a user's body to quickly absorb said bodily fluids into said absorbent portion.

22. The hygiene apparatus of claim 16, further comprising smart fabric technology.

23. The hygiene apparatus of claim 16, further comprising a thermal pack.

24. The hygiene apparatus of claim 16, further comprising an interchangeable layer, wherein said interchangeable layer further comprises a material selected from a group consisting of an endothermic and exothermic material,
whereby said material is activated when bodily fluids reach said interchangeable layer and provides a temperature change signal to a user, thereby warning said user when said bodily fluid reaches said interchangeable layer.
